(19) United States
(12) Patent Application Publication
(10) Pub. No.: US 2022/0296560 A1
(43) Pub. Date: Sep. 22, 2022

(54) PHARMACEUTICAL COMPOSITION FOR ORAL ADMINISTRATION IN POWDER FORMULATION CONTAINING ANTIVIRAL AGENT

(71) Applicant: DAE HWA PHARMA. CO., LTD., Hoengseong-gun, Gangwon-do (KR)

(72) Inventors: Jun-Hee JANG, Seoul (KR); In-Hyun LEE, Gwangju (KR); Min-Hee SON, Seongnam-si, Gyeonggi-do (KR); Hye-Jin PARK, Gwangju-si, Gyeonggi-do (KR)

(73) Assignee: DAE HWA PHARMA. CO., LTD., Hoengseong-gun, Gangwon-do (KR)

(21) Appl. No.: 17/596,585

(22) PCT Filed: Jun. 12, 2020
(86) PCT No.: PCT/KR2020/007626

§ 371 Date: Dec. 14, 2021

(30) Foreign Application Priority Data
Jun. 14, 2019 (KR) 10-2019-0070563

(51) Int. Cl. A61K 31/351; A61K 9/107; A61K 9/19; A61K 47/14; A61K 47/26

(52) U.S. Cl. CPC A61K 31/351; A61K 9/107; A61K 9/19; A61K 47/14; A61K 47/26; A61K 9/0053

(57) ABSTRACT

Provided is a pharmaceutical composition for oral administration in the form of powder obtained by a process including: (a) preparing an emulsion including zanamivir as an active ingredient; triglyceride; acyl glycerol; a nonionic surfactant; a sugar; and water; and (b) lyophilizing the emulsion prepared in the step (a). The pharmaceutical composition according to the presently claimed subject matter can significantly increase in vivo absorption rate of zanamivir. Further, the pharmaceutical composition according to the presently claimed subject matter is in the form of powder, which not only make it easy to store and distribute but also make it possible to avoid the use of functional packaging materials for preventing changes in moisture.

### TECHNICAL FIELD

The present invention relates to a pharmaceutical composition for oral administration in the form of powder comprising an antiviral agent. More specifically, the present invention relates to a pharmaceutical composition for oral administration in the form of powder obtained by lyophilizing an emulsion comprising zanamivir as an active ingredient; triglyceride; acyl glycerol; a nonionic surfactant; a sugar; and water.

### BACKGROUND ART

Zanamivir, whose chemical name is 5-(acetylamino)-4-{[amino(imino)methyl]amino}-2,6-anhydro-3,4,5-trideoxy-D-glycero-D-galacto-non-2-enonic acid, has the following chemical formula 1.

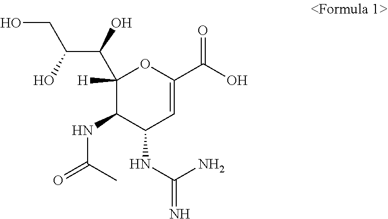 [SMILES: CC(=O)N[C@H]1[C@H]([C@H](O)[C@H](O)CO)OC(C(=O)O)=C[C@@H]1NC(=N)N]

Zanamivir exhibits the activity thereof by binding to the conserved region of the influenza virus neuraminidase to mainly catalyze the cleavage of terminal sialic acids attached to glycolipids and glycoproteins. Zanamivir is commercially available under the trade name RELENZA™. The bioavailability of zanamivir is known to be about 2% when orally administered; and that of zanamivir is known to be 4 to 17% when administered by inhalation as a powder. A powder formulation for inhalation is administered at a dose of 5 mg twice each time, twice a day for 5 days. Currently, RELENZA™, which is used for the treatment of influenza A and B infections, should be orally inhaled using a Diskhaler for administration to the respiratory tract. Therefore, an additional inhalation device for oral inhalation is required. And, the inhalation makes it difficult to administer in the same amount. In addition, there is a disadvantage of having to explain how to use the device to patients. Especially, the oral inhalation is difficult in the case of children.

The present applicant has developed a pharmaceutical composition in the form of syrup having excellent stability and content uniformity, which can improve the problem of low bioavailability of zanamivir in the oral administration thereof and solve the problems derived from a inhalation formulation (Korean Patent Application No. 10-2017-0175681, filed on Dec. 20, 2017). The pharmaceutical composition in the form of syrup according to Korean Patent Application No. 10-2017-0175681 is prepared by dissolving zanamivir at a concentration of about 5 mg/ml in a mixed solution comprising triglyceride (e.g., tricaprylin), an acyl glycerol complex (e.g., Peceol™), a nonionic surfactant (e.g., polyoxyethylene sorbitan monooleate), a sugar (sucrose) and water.

Meanwhile, since a pharmaceutical composition in the form of syrup contains a solvent (e.g., purified water), it has a problem in that it is difficult to store and distribute because it is bulky compared to a solid formulation. Especially, since a pharmaceutical composition in the form of syrup may cause stability problems such as phase separation due to a decrease in water content depending on storage conditions, it is required to use a functional packaging material that can prevent moisture change (e.g., a functional multilayer packaging film or a film having moisture-shielding function such as PVDC, EVOH, PA film).

### DISCLOSURE

### Technical Problem

The present inventors carried out various studies in order to further improve the pharmaceutical composition in the form of syrup comprising zanamivir developed by the present inventors. As the results thereof, the present inventors have developed a new formulation, that is, a pharmaceutical composition for oral administration in the form of powder to be dissolved in water (i.e., reconstituted) when used. The pharmaceutical composition for oral administration in the form of powder not only make it easy to store and distribute but also make it possible to avoid the use of functional packaging materials for preventing changes in moisture, while retaining the advantages of the pharmaceutical composition in the form of syrup comprising zanamivir developed by the present inventors.

Therefore, it is an object of the present invention to provide a pharmaceutical composition for oral administration in the form of powder comprising zanamivir.

### Technical Solution

In accordance with an aspect of the present invention, there is provided a pharmaceutical composition for oral administration in the form of powder obtained by a process comprising: (a) preparing an emulsion comprising zanamivir as an active ingredient; triglyceride; acyl glycerol; a nonionic surfactant; a sugar; and water, and (b) lyophilizing the emulsion prepared in the step (a).

In an embodiment of the pharmaceutical composition of the present invention, the step (a) may be carried out by preparing an emulsion including zanamivir in a concentration of 0.5˜mg/ml in a mixed solution comprising 1˜6% by weight of triglyceride; 1˜12% by weight of acyl glycerol; 1˜3% by weight of a nonionic surfactant; 5˜27% by weight of a sugar; and 65˜85% by weight of water.

In another embodiment of the pharmaceutical composition of the present invention, the step (a) may comprise (a1) preparing a syrup including zanamivir in a concentration of 0.5˜5 mg/ml in a mixed solution comprising 1˜20% by weight of triglyceride; 1˜30% by weight of acyl glycerol; 1˜30% by weight of a nonionic surfactant; 40˜50% by weight of a sugar; and 20˜30% by weight of water and (a2) mixing the syrup obtained in the step (a1) with water in a weight ratio of 1:2˜1:5, preferably in a weight ratio of 1:2.5˜1:4, more preferably in a weight ratio of 1:3, to prepare an emulsion.

The triglyceride may be one or more selected from the consisting of triacetin, tripropionin, tributyrin, trivalerin, tricaproin, tricaprylin, tricaprin, triheptanoin, trinonanoin, triundecanoin, trilaurin, tridecanoin, trimyristin, tripentadecanoin, tripalmitin, glyceryl triheptadecanoate, and triolein, preferably tricaprylin.

The acyl glycerol may be one or more selected from the group consisting of glyceryl behenate, glyceryl oleate, glyceryl stearate, glyceryl palmitostearate, and a complex thereof, preferably an oleoyl glycerol complex having 30 to 65% by weight of monooleoyl glycerol contents; 15 to 50% by weight of dioleoyl glycerol contents; and 2 to 20% by weight of trioleoyl glycerol contents.

The nonionic surfactant may be one or more selected from the group consisting of a polyoxyethylene-polyoxypropylene block copolymer, sorbitan fatty acid ester, polyoxyethylene sorbitan fatty acid ester, and polyoxyethylene lauryl ether, preferably polyoxyethylene sorbitan monooleate.

The sugar may be one or more selected from the group consisting of sucrose, maltose, lactose, isomaltose, fructooligosaccharide, galactooligosaccharide, isomaltooligosaccharide, maltodextrin, and mannan oligosaccharide, preferably sucrose or fructooligosaccharide.

The step (b) may further comprise freezing the emulsion obtained in the step (a) before said lyophilization.

### Advantageous Effects

The pharmaceutical composition for oral administration in the form of powder according to the present invention can be taken in the form of a transparent solution by re-dissolving it in water. In the same way as the pharmaceutical composition in the form of syrup comprising zanamivir developed by the present inventors, the pharmaceutical composition of the present invention can effectively solve the problem of low bioavailability of zanamivir, increase the drug compliance through oral administration, be applied to patients who have difficulty in inhalation, reduce the costs derived from the use of an inhalation device, and solve the problem that the dosage can be changed during the inhalation. In addition, since the pharmaceutical composition for oral administration in the form of powder according to the present invention is in the form of solid formulation, which not only make it easy to store and distribute but also make it possible to avoid the use of functional packaging materials for preventing changes in moisture.

### DESCRIPTION OF DRAWINGS

FIG. 1 shows the appearance of the syrup prepared in Example 1.

FIG. 2 shows the appearance of the pharmaceutical composition in the form of powder prepared in Example 2.

FIG. 3 shows the appearance of the formulation obtained by the reconstitution through adding purified water to the pharmaceutical composition in the form of powder obtained in Example 2.

FIG. 4 shows the appearance of the product obtained by lyophilizing the syrup obtained in Example 1 as it is (i.e., without mixing with purified water).

FIG. 5 shows the appearance of the formulation obtained by the reconstitution through adding purified water to the product obtained by lyophilizing the syrup obtained in Example 1 as it is (i.e., without mixing with purified water).

### BEST MODE

As used herein, the term “pharmaceutical composition for oral administration in the form of powder” refers to a formulation for oral administration having a powder form, which is reconstituted into the form of a solution having a clear and transparent appearance by dissolving in water when used; and then taken in the reconstituted form. The “water” used when used includes purified water, distilled water, sterile water, etc. for conventional oral administration. The “reconstitution” refers to preparing a solution having a clear and transparent appearance by adding about 0.2 to 0.3 part by weight of water such as purified water, distilled water, sterile water, etc. based on 1 part by weight of the powder formulation. Accordingly, a pharmaceutical product comprising the pharmaceutical composition of the present invention may include the above-described pharmaceutical composition for oral administration in the form of powder; and water for reconstitution, such as purified water, distilled water, sterile water, etc.

The present invention provides a pharmaceutical composition for oral administration in the form of powder obtained by a process comprising: (a) preparing an emulsion comprising zanamivir as an active ingredient; triglyceride; acyl glycerol; a nonionic surfactant; a sugar; and water, and (b) lyophilizing the emulsion prepared in the step (a).

In the pharmaceutical composition of the present invention, zanamivir used as an active ingredient may be used in a therapeutically effective amount. For example, zanamivir may be contained in an amount ranging from 0.1 to 5 mg, preferably from 1 to 5 mg, more preferably from about 1 mg, in the pharmaceutical composition for oral administration in the form of powder of the present invention, but not limited thereto.

In the pharmaceutical composition of the present invention, the step (a), a step for forming an emulsion, may be carried out by forming an emulsion by including zanamivir in the components of the pharmaceutical composition in the form of syrup disclosed in Korean Patent Application No. 10-2017-0175681, i.e., in a mixed solution comprising triglyceride, acyl glycerol, a nonionic surfactant, a sugar and water. For example, the step (a) may be carried out by preparing an emulsion including zanamivir in a concentration of 0.5˜5 mg/ml in a mixed solution comprising 1˜6% by weight, preferably 1˜4% by weight, of triglyceride; 1˜12% by weight, preferably 2˜8% by weight, of acyl glycerol; 1˜3% by weight, preferably 1˜2% by weight, of a nonionic surfactant; 5˜27% by weight of a sugar; and 65˜85% by weight of water. If necessary, the order of addition of the components in the preparation of the emulsion may be appropriately determined. For example, an emulsion may be prepared by dissolving saccharides in purified water and then dissolving zanamivir, followed by dissolving triglyceride, acyl glycerol, and a nonionic surfactant in sequence.

In addition, in the pharmaceutical composition of the present invention, the step (a) may be carried out by preparing the pharmaceutical composition in the form of syrup disclosed in Korean Patent Application No. 10-2017-0175681 and then adding water thereto to preparing an emulsion. As described in the following Examples and Experimental Examples, when the pharmaceutical composition in the form of syrup disclosed in Korean Patent Application No. 10-2017-0175681 is dried or lyophilized as it is, a sticky product is obtained and thus a formulation in the form of powder cannot be prepared. Therefore, it is preferable that the step (a) comprises (a1) preparing a syrup including zanamivir in a concentration of 0.5˜5 mg/ml in a mixed solution comprising 1˜20% by weight, preferably 3˜15% by weight, of triglyceride; 1˜30% by weight, preferably 5˜25% by weight, of acyl glycerol; 1˜30% by weight, preferably 2˜25% by weight, of a nonionic surfactant; 40˜50% by weight of a sugar; and 20˜30% by weight of water and (a2) mixing the syrup obtained in the step (a1) with water in a weight ratio of 1:2˜1:5 to prepare an emulsion. If necessary, the order of addition of the components in the preparation of the emulsion in the step (a1) may be appropriately determined. For example, an emulsion may be prepared by dissolving a sugar in purified water, dissolving zanamivir, and then dissolving triglyceride, acyl glycerol, and a nonionic surfactant in sequence, followed by the step (a2).

The triglyceride may be one or more selected from the consisting of triacetin, tripropionin, tributyrin, trivalerin, tricaproin, tricaprylin, tricaprin, triheptanoin, trinonanoin, triundecanoin, trilaurin, tridecanoin, trimyristin, tripentadecanoin, tripalmitin, glyceryl triheptadecanoate, and triolein, preferably tricaprylin [e.g., Captex™ 8000 (Abitec)].

The acyl glycerol, unless otherwise indicated, includes monoacyl glycerol, diacyl glycerol, triacyl glycerol, or a complex thereof. For example, the acyl glycerol may be one or more selected from the group consisting of glyceryl behenate, glyceryl oleate, glyceryl stearate, glyceryl palmitostearate, and a complex thereof. Preferably, the acyl glycerol may be an oleoyl glycerol complex having 30 to 65% by weight of monooleoyl glycerol contents; 15 to 50% by weight of dioleoyl glycerol contents; and 2 to 20% by weight of trioleoyl glycerol contents. In an embodiment, the oleoyl glycerol complex may be an oleoyl glycerol complex having 32 to 52% by weight of monooleoyl glycerol contents; 30 to 50% by weight of dioleoyl glycerol contents; and 5 to 20% by weight of trioleoyl glycerol contents [e.g., Peceol™ (Gattefosse)]. In another embodiment, the oleoyl glycerol complex may be an oleoyl glycerol complex having 55 to 65% by weight of monooleoyl glycerol contents; 15 to 35% by weight of dioleoyl glycerol contents; and 2 to 10% by weight of trioleoyl glycerol contents [e.g., CAPMUL™ (Abitec)].

The nonionic surfactant may be one or more selected from the group consisting of a polyoxyethylene-polyoxypropylene block copolymer, sorbitan fatty acid ester, polyoxyethylene sorbitan fatty acid ester, and polyoxyethylene lauryl ether, preferably polyoxyethylene sorbitan monooleate [e.g., Tweeen™ 80 (NOF)].

The sugar may be one or more selected from the group consisting of sucrose, maltose, lactose, isomaltose, fructooligosaccharide, galactooligosaccharide, isomaltooligosaccharide, maltodextrin, and mannan oligosaccharide, preferably sucrose or fructooligosaccharide.

In an embodiment, the step (a) may be carried out by preparing an emulsion including zanamivir in a concentration of 0.5˜5 mg/ml in a mixed solution comprising 1˜6% by weight of tricaprylin; 1˜12% by weight of an oleoyl glycerol complex; 1˜3% by weight of polyoxyethylene sorbitan monooleate; 5˜27% by weight of sucrose or fructooligosaccharide; and 65˜85% by weight of water.

In another embodiment, the step (a) may comprise (a1) preparing a syrup including zanamivir in a concentration of 0.5˜5 mg/ml in a mixed solution comprising 1˜20% by weight of tricaprylin; 1˜30% by weight of an oleoyl glycerol complex; 1˜30% by weight of polyoxyethylene sorbitan monooleate; 40˜50% by weight of sucrose or fructooligosaccharide; and 20˜30% by weight of water and (a2) mixing the syrup obtained in the step (a1) with water in a weight ratio of 1:2˜1:5, preferably in a weight ratio of 1:2.5˜1:4, more preferably in a weight ratio of about 1:3, to prepare an emulsion.

In the pharmaceutical composition of the present invention, the step (b) may further comprise freezing the emulsion obtained in the step (a). That is, the step (b) may be carried out by freezing the emulsion obtained in step (a) and then lyophilizing the resultant. It is preferable that the freezing is performed by rapid freezing, so as to maintain a uniform dispersion state of the emulsion. The lyophilization may be carried out with a lyophilizer according to a method conventionally used in the field of pharmaceutics, for example, at a temperature of about −114° C. or less and at a pressure of 10 mTorr or less for 2 to 8 hours, preferably for about 5 hours.

The present invention will be described in further detail with reference to the following examples and experimental examples. These examples and experimental examples are for illustrative purposes only and are not intended to limit the scope of the present invention.

### EXAMPLE 1: Preparation of a Syrup

A zanamivir-containing syrup was prepared according to the components and amounts shown in Table 1. Sucrose was dissolved in purified water and then zanamivir was added thereto. The mixture was transparently dissolved by stirring at 1,000 rpm at 25° C. for 2 hours. Captex™ 8000 (Abitec), Peceol™ (Gattefosse) and Tweeen™ 80 (NOF) were sequentially added thereto, followed by stirring at 1,000 rpm to prepare a syrup.

TABLE 1

| | Components of Excipients (% by weight) | | | | |
|---|---|---|---|---|---|
| Zanamivir | | Purified | Captex ™ | | |
| (concentration) | Sucrose | water | 8000 | Peceol ™ | Tweeen ™ |
| 1 mg/ml | 44.78 | 22.35 | 9.13 | 18.27 | 5.47 |

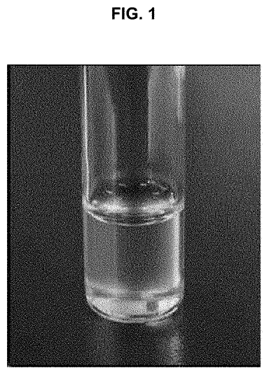

The appearance of the obtained syrup is shown in FIG. 1. As shown in FIG. 1, the resulting syrup exhibited a clear and transparent appearance.

### EXAMPLE 2: Preparation of a Pharmaceutical Composition For Oral Administration in the Form of Powder

The syrup obtained in Example 1 was mixed with purified water in a weight ratio of 1:3 to form an emulsion, rapidly frozen using liquid nitrogen, and then lyophilized at the temperature of −114° C. or less and at the pressure of 10 mTorr or less for about 5 hours to prepare 7.871 g of a pharmaceutical composition in the form of powder.

### EXAMPLE 3: Preparation of a Pharmaceutical Composition For Oral Administration in the Form of Powder

4.48 g of Sucrose was dissolved in 22.24 g of purified water and then 0.03 g of zanamivir was added thereto. The mixture was transparently dissolved by stirring at 1,000 rpm at 25° C. for 2 hours. 0.91 g of Captex™ 8000 (Abitec), 1.83 g of Peceol™ (Gattefosse) and 0.55 g of Tweeen™ 80 (NOF) were sequentially added thereto, followed by stirring at 1,000 rpm to form an emulsion (30.1 ml). The emulsion was rapidly frozen using liquid nitrogen and then lyophilized at the temperature of −114° C. or less and at the pressure of 10 mTorr or less for about 5 hours to prepare 7.771 g of a pharmaceutical composition in the form of powder.

### Experimental Example 1: Absorption Rate Tests of the Syrup

The syrup prepared in Example 1 was orally administered to ICR mice (6 weeks old, female) at the doses of 50 mg/kg and 100 mg/kg as zanamivir, using a gastric sonde, respectively. After the administrations, blood samples were collected from the orbital vein of each mouse at 0, 30 minutes, 1, 2, and 4 hours and then centrifuged at 8,000×g at 4° C. for 20 minutes to obtain plasma samples, which were stored at −70° C. The plasma samples were melted at room temperature and then stirred for 1 minute with a vortex mixer. 200.0 μL of 70% acetonitrile and 300.0 μL of 60% acetonitrile were added to 100.0 μL of each plasma sample, which was then stirred for 5 minutes at 3,000 rpm using a vortex mixer. After centrifuging each sample at 14,000×g, 4° C. for 20 minutes, 300.0 μL of the supernatant was taken therefrom and then filtered using a syringe filter (PTFE, chromdisc, 13 mm, pore size 0.20 mm). 200.0 μL of the filtrate was taken and then analyzed by HPLC under the following conditions.

<HPLC Conditions >
Column: 4.6 mm×250 mm, 5 μm, NH2p-50
Column temperature: 30° C.
Flow rate: 1 ml/min
Detector: UV 234 nm
Injection volume: 60 μl
Mobile phase: acetonitrile: purified water=65:35 (v/v)

The pharmacokinetic parameters calculated from each blood concentration profile of zanamivir measured as described above are shown in Table 2 below.

TABLE 2

| Dose | | Cmax (μg/ml) | Tmax (hr) | AUC_{0-4 hr} (min · μg/ml) |
|---|---|---|---|---|
| 50 | mg/kg | 0.36 ± 0.013 | 0.5 | 55.8 |
| 100 | mg/kg | 0.71 ± 0.15 | 0.5 | 78 |

As shown in Table 2, it can be seen that the Cmax and AUC₀₋₄ₕᵣ values increase as the oral dose increases.

### Experimental Example 2: Stability Tests of the Syrup

The zanamivir-containing syrup prepared in Example 1 was stored at 25° C. for 0 and 40 days, followed by measuring the appearances and contents thereof. The results are shown in Table 3 below.

TABLE 3

| | |
|---|---|
| Storage period | Contents of zanamivir (% by weight) |

| | | |
|---|---|---|
| 0 | Day | 97.4 ± 0.18 |
| 40 | Days | 96.6 ± 0.57 |

As shown in Table 3, as a result of the content test evaluation of the syrup, it can be confirmed that the content of the syrup was maintained after 40 days of storage, compared to the initial formulation, and thus that the syrup is stable. In addition, phenomena such as layer separation and precipitation were not observed in both the is initial syrup and the syrup after storage, which confirms that the syrup is stable.

### Experimental Example 3: Evaluation on the Forming-Abilities of Powder Formulations

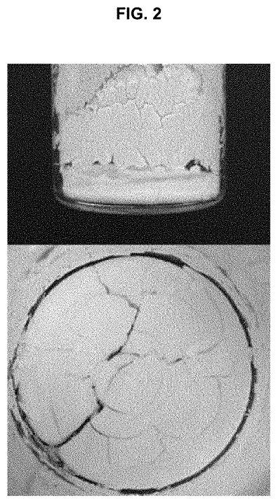

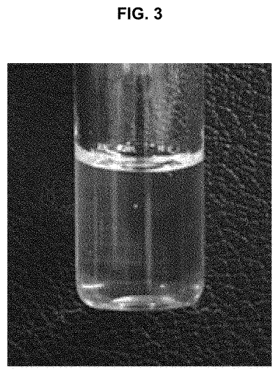

The appearance of the pharmaceutical composition in the form of powder obtained in Example 2 is shown in FIG. 2. In addition, the appearance of the formulation obtained by the reconstitution thereof, i.e., through adding 2.26 ml of purified water to the pharmaceutical composition in the form of powder obtained in Example 2 (7.871 g), is shown in FIG. 3. As can be seen from the results of FIGS. 2 and 3, the pharmaceutical composition obtained according to the present invention was in the form of powder; and provided a clear and transparent syrup when reconstituted with water.

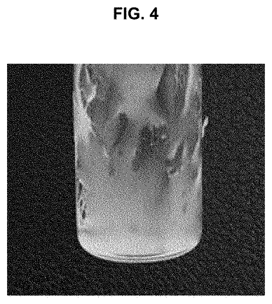

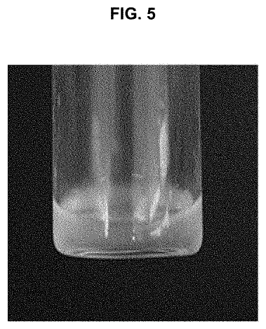

In addition, the syrup obtained in Example 1 was rapidly frozen using liquid nitrogen as it is (that is, without mixing with purified water) and then lyophilized under the same conditions as in Example 2. The appearance of the resulting product is shown in FIG. 4. In addition, the appearance of the formulation obtained by the reconstitution thereof, i.e., through adding 2.26 ml of purified water to the resulting product (7.74 g), is shown in FIG. 5. As can be seen from the results of FIGS. 4 and 5, when the syrup was lyophilized without an additional treatment (i.e., without mixing with water), it exhibited a sticky paste appearance and provided an opaque syrup when reconstituted with water.

### Experimental Example 4: Stability Tests of the Powder Formulation

The powder formulation prepared in Example 2 was stored at 25° C. for 0 and 40 days, followed by measuring the appearances and contents thereof. The results are shown in Table 4 below.

TABLE 4

| | |
|---|---|
| Storage period | Contents of zanamivir (% by weight) |

| | | |
|---|---|---|
| 0 | Day | 100.10 ± 0.57 |
| 40 | Days | 100.06 ± 0.36 |

As shown in Table 4, as a result of the content test evaluation of the powder formulation, it can be confirmed that the formulation after the storage period of 40 days maintained the content thereof, compared to the initial formulation, which confirms that the formulation is stable.

1. A pharmaceutical composition for oral administration in the form of powder obtained by a process comprising:
(a) preparing an emulsion comprising zanamivir as an active ingredient; triglyceride; acyl glycerol; a nonionic surfactant; a sugar; and water, and
(b) lyophilizing the emulsion prepared in the step (a).

2. The pharmaceutical composition according to claim 1, wherein the step (a) is carried out by preparing an emulsion including zanamivir in a concentration of 0.5˜5 mg/ml in a mixed solution comprising 1˜6% by weight of triglyceride; 1˜12% by weight of acyl glycerol; 1˜3% by weight of a nonionic surfactant; 5˜27% by weight of a sugar; and 65˜85% by weight of water.

3. The pharmaceutical composition according to claim 1, wherein the step (a) comprises (a1) preparing a syrup including zanamivir in a concentration of 0.5˜5 mg/ml in a mixed solution comprising 1˜20% by weight of triglyceride; 1˜30% by weight of acyl glycerol; 1˜30% by weight of a nonionic surfactant; 40˜50% by weight of a sugar; and 20˜30% by weight of water and (a2) mixing the syrup obtained in the step (a1) with water in a weight ratio of 1:2˜1:5 to prepare an emulsion.

4. The pharmaceutical composition according to claim 1, wherein the triglyceride is one or more selected from the consisting of triacetin, tripropionin, tributyrin, trivalerin, tricaproin, tricaprylin, tricaprin, triheptanoin, trinonanoin, triundecanoin, trilaurin, tridecanoin, trimyristin, tripentadecanoin, tripalmitin, glyceryl triheptadecanoate, and triolein.

5. The pharmaceutical composition according to claim 4, wherein the triglyceride is tricaprylin.

6. The pharmaceutical composition according to claim 1, wherein the acyl glycerol is one or more selected from the group consisting of glyceryl behenate, glyceryl oleate, glyceryl stearate, glyceryl palmitostearate, and a complex thereof.

7. The pharmaceutical composition according to claim 6, wherein the acyl glycerol is an oleoyl glycerol complex having 30 to 65% by weight of monooleoyl glycerol contents; 15 to 50% by weight of dioleoyl glycerol contents; and 2 to 20% by weight of trioleoyl glycerol contents.

8. The pharmaceutical composition according to claim 1, wherein the nonionic surfactant is one or more selected from the group consisting of a polyoxyethylene-polyoxypropylene block copolymer, sorbitan fatty acid ester, polyoxyethylene sorbitan fatty acid ester, and polyoxyethylene lauryl ether.

9. The pharmaceutical composition according to claim 8, wherein the nonionic surfactant is polyoxyethylene sorbitan monooleate.

10. The pharmaceutical composition according to claim 1, wherein the sugar is one or more selected from the group consisting of sucrose, maltose, lactose, isomaltose, fructooligosaccharide, galactooligosaccharide, isomaltooligosaccharide, maltodextrin, and mannan oligosaccharide.

11. The pharmaceutical composition according to claim 10, wherein the sugar is sucrose or fructooligosaccharide.

12. The pharmaceutical composition according to claim 2, wherein the step (a) is carried out by preparing an emulsion including zanamivir in a concentration of 0.5˜5 mg/ml in a mixed solution comprising 1˜6% by weight of tricaprylin; 1˜12% by weight of an oleoyl glycerol complex; 1˜3% by weight of polyoxyethylene sorbitan monooleate; 5˜27% by weight of sucrose or fructooligosaccharide; and 65˜85% by weight of water.

13. The pharmaceutical composition according to claim 3, wherein the step (a) comprises (a1) preparing a syrup including zanamivir in a concentration of 0.5˜5 mg/ml in a mixed solution comprising 1˜20% by weight of tricaprylin; 1˜30% by weight of an oleoyl glycerol complex; 1˜30% by weight of polyoxyethylene sorbitan monooleate; 40˜50% by weight of sucrose or fructooligosaccharide; and 20˜30% by weight of water and (a2) mixing the syrup obtained in the step (a1) with water in a weight ratio of 1:2˜1:5 to prepare an emulsion.

14. The pharmaceutical composition according to claim 3, wherein the step (a2) is carried out by mixing the syrup obtained in the step (a1) with water in a weight ratio of 1:2.5˜1:4 to prepare an emulsion.

15. The pharmaceutical composition according to claim 14, wherein the step (a2) is carried out by mixing the syrup obtained in the step (a1) with water in a weight ratio of 1:3 to prepare an emulsion.

16. The pharmaceutical composition according to claim 1, wherein the step (b) further comprises freezing the emulsion obtained in the step (a) before said lyophilization.
